(19) United States
(12) Patent Application Publication
(10) Pub. No.: US 2014/0017304 A1
(43) Pub. Date: Jan. 16, 2014

(54) METHOD FOR ENCAPSULATED THERAPEUTIC PRODUCTS AND USES THEREOF

(71) Applicants: Myriam Bosmans, Nieuwenrode (BE); Luc Schoonjans, Herent (BE); Gudmund Skjäk-Braek, Trondheim (NO)

(72) Inventors: Myriam Bosmans, Nieuwenrode (BE); Luc Schoonjans, Herent (BE); Gudmund Skjäk-Braek, Trondheim (NO)

(21) Appl. No.: 14/008,290

(22) PCT Filed: Mar. 7, 2012
(86) PCT No.: PCT/EP2012/053868

§ 371 Date: Sep. 27, 2013

(30) Foreign Application Priority Data
Mar. 29, 2011 (EP) 11160298.3

(51) Int. Cl. A61K 35/39; A61J 3/00; A61K 47/36

(52) U.S. Cl. CPC A61K 35/39; A61K 47/36; A61J 3/00
USPC 424/451; 424/93.7; 264/4.1

(57) ABSTRACT

The current invention relates to encapsulation methods comprising alginate-based microencapsulation for the immune-protection and long-term functioning of biological material or therapeutics. The biological material or the therapeutics are encompassed by a membrane formed by jellifying an alginate polymer. Specifically, although by no means exclusively, the encapsulation system is intended for use in allo-or xenotransplantation. The membrane provides for a protective barrier of the encapsulated material, ensuring the longevity and preventing unwanted influences from outside the barrier, such as inflammatory reactions or immune-responses. The invention is furthermore directed to methods of producing and providing the encapsulated products for use in cell therapies. The therapeutic products obtained by the encapsulation method may provide a method for ameliorating of treating a range of conditions.

### TECHNICAL FIELD

The invention relates to encapsulation methods comprising alginate-based microencapsulation for the immune-protection and long-term functioning of living cells or therapeutics. Specifically, although by no means exclusively, the encapsulation system is for use in allo- and xeno- transplantation. The invention is also directed to methods of making and using the encapsulation system and the use of encapsulated cell products in cell therapies.

### BACKGROUND

Cell transplantation is becoming increasingly more successful both experimentally and clinically. It takes advantage of developments in material science, cell biology, and drug delivery to develop micro- and macro-encapsulated cell therapy platforms. These constructs allow for the controlled delivery of therapeutic molecules for the treatment of acute and chronic diseases, but their widespread use is hampered by the need for frequent administration for erodible materials, and retrieval and chronic biocompatibility issues for non-degradable materials. In the case of biodegradable materials, the success of encapsulated cell therapy will depend to a large degree on an understanding of the stability of the material once transplanted and ultimately how that stability impacts the ability of the graft to support cell survival, protein secretion and diffusion, immune-isolation, biocompatibility, physical placement and fixation, degradation, and the efficacy and pharmacodynamics of the secreted product. Cell (micro) encapsulation is a well-established concept that can be implemented for many applications, such as cell therapy, cell biosensors, cell immobilization for protein and antibody production, probiotic encapsulation by the food industry or nutraceutics. Cell therapy, which is the use of living cells to treat pathological conditions, could be a solution to the difficulties encountered in therapeutic protein delivery. Indeed, the production and administration of proteins are challenging because of their physicochemical and biological characteristics.

Micro-encapsulation is the process in which small, discrete substances from for instance biological origin become enveloped by a membrane which is preferably compatible with the recipient in which it is placed.

The produced membrane is semi-permeable which permits the influx of molecules essential for cell metabolism (nutrients, oxygen, growth factors, etc.) and outward diffusion of therapeutic proteins and waste products. At the same time, cells and larger molecules of the immune system are kept away, avoiding lifelong exposure to highly toxic immunosuppressant drugs. Many device types have been proposed, but embedding in a matrix displays significant advantages as such devices optimize mass transfer because of high surface vs. internal volume ratios, which is critical for cell viability and fast secretory responses to external signal. Although such artificial devices are not directly connected to the host body and organs (extravascular devices), they have been shown to support entrapped cell metabolism, growth, and differentiation. Moreover, the deposition of matrix embedded substances in specific body compartments achieves high, sustained, local concentration of proteins, decreasing potential side-effects. Matrices and hollow spheres can be produced efficiently by many techniques well described for drug delivery and other non-pharmacological applications. However, in cell encapsulation applications, complex and conflicting requirements have to be met. Not only are very reproducible methods needed for the preparation of devices with very precise parameters (permeability, size, surface), but also these methods should additionally support cell integrity and viability during the encapsulation process and after implantation. Finally, the preparation method must ensure adequate flux across the particle membrane for cell survival and function as well as long-term biocompatibility with host tissues without associated inflammatory reactions (incl. effective neovascularization).

While the attempts to transplant such encapsulated material into a patient to perform the specific function of that material inside the recipient patient have been partially successful, the patient's body often reacts in ways that impair the activity of the devices by fibroblast or other inflammation-related overgrowth of this substance by the body. A potential mechanism for the induction of fibroblasts is the activation of macrophages, and the resultant stimulation of cytokines by the particle substance. Cytokines are molecules secreted by the body in response to a new set of antigens, and are often toxic to the encapsulated cells. Some cytokines in turn stimulate the immune system of the patient. Thus, immune response can still be a limiting factor in the effective life of the encapsulated material. In addition, fibroblast cells tend to overgrow the devices, also apparently in response to the newly released cytokines. This growth of fibroblasts causes the devices to lose their porosity. As a result, the cellular material inside the devices cannot receive nutrients and the product of the cellular material cannot permeate the device wall. This can cause the encapsulated living material to die, and can impair the effectiveness of the devices as a delivery system.

The nature of the biomaterial is crucial for the viability of the transplanted devices. Various biocompatible materials are described to be suitable for their use in encapsulating cells. Examples are for instance agar, alginate, carrageenan, cellulose and its derivatives, chitosan, collagen, gelatin, epoxy resin, photo cross-linkable resins, polyacrylamide, polyester, polystyrene and polyurethane, polyethylene glycol (PEG).

Extensive work has been done using alginate which is regarded as a highly efficient biomaterial for cell microencapsulation. Alginate is a natural polymer, which can be extracted from algae.

Alginate comprises a heterogeneous group of linear binary copolymers of 1-4 linked β-D-mannuronic acid and its C-5 epimer α-L-guluronic acid. Alginate has long been studied as a biomaterial in a wide range of physiologic and therapeutic applications. Its potential as a biocompatible implant material was first explored in 1964 in the surgical role of artificially expanding plasma volume (Murphy et al., Surgery. 56: 1099-108, 1964). Over the last twenty years, there has been remarkable progress in alginate cell microencapsulation for the treatment of diseases such as diabetes amongst others.

Despite success in numerous animal models and in clinical allo-transplantation, there have been variable degradation kinetics impacting diffusion, immune-isolation, and ultimately leading to loss of graft survival and rejection. The general understanding of the stability of alginate particles in vivo from a strict materials perspective is limited and this in turn limits their use.

Although some attempts have been made to optimize the performance of the particles by improving their biocompatibility and stability (see, for example, Sun et al., (1987)), relatively little has been done to correlate the molecular structure and size of the main polymer component of the particles, the alginate, to the functional properties of the resulting particle.

Several patents and patent applications have attempted to perfect the materials and methods of encapsulation:

WO 91/09119 discloses a method of encapsulating biological material, more specifically islet cells, in a bead with an alginate gel, which is subsequently encapsulated by a second layer, preferentially poly-L-lysine, and a third layer consisting of alginate.

U.S. Pat. No. 5,084,350 provides a method for encapsulating biologically active material in a large matrix, which is subsequently followed by liquification of the microcapsules.

U.S. Pat. No. 4,663,286 discloses a method of making microcapsules by jelling the microcapsule, and subsequent expanding the microcapsule by hydration to control the permeability of the capsule.

Prior art capsules suffer from several problems which affect their longevity, since the requirement for liquification of the core compromises the structural integrity of the capsule. In addition, dejellying is a harsh treatment for living cells. Furthermore, a poly-lysine coating, which if exposed can cause fibrosis, is not as tightly bound to the calcium alginate inner layer as it could be. Moreover, dejellying of the capsule core may result in the leaching out of unbound poly-lysine or solubilized alginate, causing a fibrotic reaction to the microcapsule. Furthermore, the shape and structure of the device equally plays a role in the viability of the encapsulated biological material after implantation. A significant complication arising from encapsulated systems is the decreased efficiency by which oxygen, nutrients and metabolic waste diffuse in and out the device. Spheres tend to form large aggregates within a body cavity and hence, the cells in the center of these aggregates are more prone to cell death and necrosis, due to a lack of nutrients. Eventually, the envisaged effect of the implant will be seriously reduced or even lost. The present invention is aimed to overcome at least part of the above-mentioned problems in prior art.

### SUMMARY OF THE INVENTION

It is an object of the present invention to improve the stability of the alginate-based bio-devices and to produce therapeutic products based on these bio-devices which can be used for in vivo applications.

Compared with prior art alginate polycation capsules, the encapsulation procedures of the present invention display several improved characteristics, i.e., (i) higher mechanical and chemical stability, (ii) causes no or very low inflammatory reaction in the recipient (iii) allows low impact surgical procedures for implantation, (iv) reinforces the durability of the microdevices after implantation by reducing the risk of necrosis.

The alginate-based encapsulation of the present invention (having improved mechanical and chemical stability and biocompatibility) is made by selecting the material to be used for encapsulating (and the gelling ions therefor) according to the desired chemical structure and molecular sizes, as well as by controlling the kinetics of matrix formation. Invention devices are preferably made from guluronic acid enriched alginate. The device is further characterized by a defined ratio of calcium/barium alginates. Various shapes of alginate devices can be produced. In a preferred embodiment the device consists of a filamentous shape. By using encapsulated cells in a filamentous form, the longevity of the implant is ensured. The inventors have found that implanting the microparticles in a filamentous form has the advantage that the encapsulated cells are less prone to cell death and necrosis, as the filaments do not tend to form large aggregates after implantation, as other shapes in prior art are known to do. Formation of large aggregates impairs the influx of nutrients to the inner cells of the aggregate, which causes starvation and eventually loss of these inner cells. The filaments can furthermore be more easily handled and surgically or laparoscopically transplanted by the surgeons in sites other than the peritoneum such as, but not limited to fat, the omentum or subcutaneous sites. In case of clinical complications they might also be easier removed than the common alginate capsules.

Unlike many prior art devices, there is no dejellying of the alginate core of invention particles as this impairs cell viability.

Also, because, in a preferred embodiment, the inner core alginate is made of barium and calcium ionically cross-linked alginate, it is more stable than prior art calcium alginate, and less toxic than prior art barium alginate.

While barium has the stronger affinity, it is toxic in large amounts, and therefore, creates a safety hazard that is undesirable. It has, however, in accordance with the present invention, been unexpectedly found that a combination of barium and calcium, within a particular concentration range, has the benefits of high affinity without the disadvantages of a high risk of toxicity.

### DESCRIPTION OF FIGURES

FIG. 1. Non-fasting blood glucose levels in diabetic Nod/Scid mice treated with 2.9M encapsulated human beta cells compared to non-treated diabetic animals and non-treated non-diabetic controls. The data represent means (wherever appropriate) ±SD.

FIG. 2. Human C-peptide levels in experimental and control groups after implantation of 2.9M encapsulated human beta cells in diabetic Nod/Scid mice.

FIG. 3. Effect of treatment on the body weight of mice.

FIG. 4. Example of the type of nozzle used to obtain encapsulated cells in the form of filaments.

### DETAILED DESCRIPTION OF THE INVENTION

The present invention concerns an encapsulation system for living cells and therapeutics which has improved bio-stability when the encapsulated cells and therapeutics are implanted into a recipient. This improved formulation enables the encapsulated cells and therapeutics to remain functional within a living body for longer periods than is currently the case which result in improved therapeutic delivery and thus treatment efficacy.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the term biological material includes DNA, RNA, proteins, organelles, antibodies, immuno-proteins, peptides, hormones, viable tissue or viable prokaryotic or eukaryotic cells.

As used herein, the term biocompatible matrix comprises a compound selected from the group of agar, alginate, carrageenan, cellulose and its derivatives, chitosan, collagen, gelatin, epoxy resin, photo cross-linkable resins, polyacrylamide, polyester, polystyrene and polyurethane, polyethylene glycol (PEG).

As used herein, the term alginate-conjugates can include, but are not limited to, alginate-collagen, alginate-laminin, alginate-elastin, alginate-fibronectin, alginate-collagen-laminin and alginate-hyaluronic acid in which the collagen, laminin, elastin, collagen-laminin or hyaluronic acid is covalently bonded (or not bonded) to alginate.

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise,” “comprising,” and “comprises” and “comprised of” as used herein are synonymous with “include”, “including”, “includes” or “contain”, “containing”, “contains” and are inclusive or open-ended terms that specifies the presence of what follows component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

The expression “% by weight” (weight percent), here and throughout the description unless otherwise defined, refers to the relative weight of the respective component based on the overall weight of the formulation.

In a first aspect, the invention provides for an encapsulation system comprising alginate which is high in guluronic acid. Alginate is a linear polysaccharide consisting of (1→4)-linked β-D-mannuronate (M) and its C-5 epimer α-L-guluronate (G). The monomers can appear in homopolymeric blocks of consecutive G-residues (G-blocks), consecutive M-residues (M-blocks), alternating M and G-residues (MG-blocks) or randomly organized blocks. Since the purity degree of the alginate has been shown to determine the biocompatibility of alginate based particles it is mandatory to provide details of the purity. According to FDA requirements for device implantation the content of endotoxin must be below 350 EU per patient (below 15 EU for CNS applications). As the chemical properties of endotoxins are very similar to alginates, their removal has been a challenging task but purified alginates with a specified endotoxin content below 100 EU/g are now commercially available. GMP requires that the alginates are characterized by validated methods according to ASTM guide 2064. Per batch a certificate should be delivered. The present invention provides a composition comprising a high guluronic acid alginate, with a guluronic acid content of at least 60% and cations.

In a preferred embodiment of the invention, the biocompatible alginate-based matrices prepared using the encapsulation methodology combines a micro-droplet generator and a gelling buffer to encapsulate the biological material of interest in inhomogeneous alginate-Ca2+/Ba2+ microparticles. Upon extrusion through a micro-droplet generator droplets are produced by a combination of air shears and mechanical pressure by a peristaltic pump. Alternatively an electrostatic bead generator can be used to produce the droplets. The biological material containing micro-droplets are subsequently collected into a cationic cross-linking solution with buffer (pH 7.2-7.4). When brought in contact with this buffer the micro-droplets jellify. The cationic cross-linking agent may be selected from salts of the group consisting of Ag⁺, Al³⁺, Ba²⁺, Ca²⁺, Cd²⁺, Cu²⁺, Fe²⁺, Fe3+, H⁺, K⁺, Li⁺, Mg²⁺, Mn²⁺, Na³⁰ , NH₄⁺, Ni²⁺, Pb²⁺, Sn²⁺ and Zn²⁺. Preferably the cationic cross-linking agent is a combination of barium chloride and calcium chloride. The cross-linking agent is preferably in excess, for example from 1 mM to 20 mM barium chloride and from 1 mM to 20 mM calcium chloride. More preferably 10 mM barium chloride and 10 mM calcium chloride.

Thereafter, micro-droplets are washed three times with Ringer's Solution and maintained in serum free Ham's F-10 medium at 37° C. and 5% CO2 until transplantation. Micro-droplet size varies between 200-800 μm. The micro-droplets may take many forms, such as granules, spheres, sheets or filamentous structures. In a most preferred embodiment, the micro-droplets take the form of alginate-based filaments by using a slightly modified procedure.

The formed micro-droplets swell approximately 10% or greater in volume when placed in vitro in physiological conditions for about one month or more. Swelling of these alginate matrices is thought to be caused by surplus divalent cations causing an osmotic gradient leading to water uptake. The spheres and filaments of the invention are highly stable. It is expected that the micro-droplets of the present invention will be able to remain functional in vivo in a subject for a significant period of time and certainly for periods up to 4 months and more.

In a further preferred embodiment, the encapsulated biological material comprises of cells, such as, but not limited to islet cells, hepatocytes, neuronal cells, pituitary cells, chromaffin cells, chondrocytes, germ line cells and cells that are capable of secreting factors. The cells are processed according to appropriate methods (e.g. for islet cells the method described in EP1146117 and related) and are mixed with a 1.8% sterile ultrapure alginate solution to obtain a final cell density between 10-30×10⁶ cells/mL alginate.

In a particularly preferred embodiment, the encapsulated biological material comprises a pool of pancreatic, endocrine cells that originate from immature porcine pancreas, capable of secreting insulin, useful for the treatment of diabetes. The cells may alternatively comprise hepatocyte or non-hepatocyte cells capable of secreting liver secretory factors useful in the treatment of liver diseases or disorders. The cells may alternatively comprise neuronal cells, such as choroids plexus, pituitary cells, chromaffin cells, chondrocytes and any other cell capable of secreting neuronal factors useful in the treatment of neuronal diseases such as Parkinson's disease, Alzheimer's disease, epilepsy, Huntington's disease, stroke, Reiter neuron disease, amyotrophic lateral sclerosis (ALS), multiple sclerosis, aging, vascular disease, Menkes Kinky Hair Syndrome, Wilson's disease, trauma or injury to the nervous system.

In another preferred embodiment the encapsulated biological material may be genetically engineered cells producing therapeutic proteins such as, but not limited to erythropoietin, insulin, IGF-1, IL-2, cytochrome P450, CNTF, NGF, BMPs, BDNF, GDNF, VEGF, blood clotting factors, interferons, dopamine, endostatin, neuropilin-1, GH3 and antibodies.

In another embodiment, the encapsulated biological material might comprise stem cells or progenitor cells. Stem and progenitor cells have the potency to differentiate into various cell lineages and hence hold a huge potential in cellular therapy in regenerative medicine. However, failure of tissue regeneration and remodelling is partly attributed to the lack of protection of the stem and progenitor cells to extrinsic factors. Microencapsulation can immobilize stem cells to provide a favourable microenvironment for the stem cells survival and functioning, hence creating a bio-artificial stem cell niche which mimics specific physicochemical and biochemical characteristics of the normal stem cell niche.

The invention furthermore provides a method of ameliorating or treating a disease or condition in an animal, including a human, comprising transplanting an effective amount of the cell-containing alginate matrices of the invention into said animal, wherein said cells secrete a therapeutic that is effective at ameliorating or treating said disease or condition.

The invention further provides a method of ameliorating or treating a disease or condition in an animal, including a human, comprising transplanting an effective amount of the cell-containing immuno-protective membrane coated non-degradable cell delivery construct of the invention into said animal, wherein said cells secrete a therapeutic that is effective at ameliorating or treating said disease or condition.

The invention further provides a method of ameliorating or treating a disease or condition in an animal, including a human, comprising transplanting an effective amount of the therapeutic-containing alginate matrices of the invention into said animal, wherein said therapeutic is effective at ameliorating or treating said disease or condition.

In these methods of treatment, the matrices or coated delivery constructs of the invention may be administered in an amount that would deliver sufficient therapeutic so as to be effective against the disease. For example, in the treatment of diabetes, a minimum amount of one million encapsulated insulin producing cells per kilogram bodyweight of the recipient is implanted.

A skilled practitioner would be able to test the secretion rate of the particular therapeutic from the alginate matrices in vitro and, for any particular patient need, be able to calculate how many spheres or filaments would be required to treat that particular patient effectively.

The matrices of the invention may be formulated for allo- or xeno- transplantation depending on the source of the living cells and/or therapeutics. The matrices of the invention may be transplanted within the tissues of the body or within fluid-filled spaces of the body, whichever is the most appropriate in terms of accessibility and efficacy. More specifically, the implantation or transplantation site may be subcutaneous, intramuscular, intra-organ, intravenous, arterial/venous vascularity of an organ, cerebrospinal fluid, and lymphatic fluid. For example, if the living cells within the matrices are beta cells, they may be transplanted in the peritoneal cavity. In preferred embodiment, the encapsulated cells are implanted into the omentum, a highly vascularized structure within the peritoneal cavity. In case of safety issues with the alginate matrices, a straightforward omentectomy can be performed, safely removing the matrices. Other implantation sites include fat and subcutaneous sites. Again, in case of clinical complications they might be easily removed.

In one embodiment, the devices may be provided in an injectable form, which allows a straightforward implantation or transplantation. Alternatively, the devices may be formulated for oral or topical administration, particularly when they contain a therapeutic bioactive agent, such as an antibiotic.

The present invention will be now described in more details, referring to examples that are not limitative.

### EXAMPLES

### Exmple 1

### Human Islets Encapsulated in Alginate Microparticles—Normalization in Mice

A coaxial airflow device (a microdroplet generator) in combination with a Barium/Calcium gelling buffer, is used to encapsulate the human pancreatic islets in inhomogeneous alginate-Ca2+/Ba2+ microparticles.

a) Cell preparation before encapsulation
the human islet suspension is centrifuged at 270 g (1100 RPM in Beckman GS-6R); 3 min; 15-30° C.
the supernatant (Ham F10) is removed.
the cells are washed twice with NaCl 0.9% with intermediate centrifugation: 270 g (1100 RPM in Beckman GS-6R); 3 min; 15-30° C.
the cell pellet is gently mixed with alginate 1.8% using a pipet until homogeneous suspension is obtained. Human islets are mixed with a 1.8% sterile ultrapure alginate solution to obtain a final cell density between 5-50×10⁶ cells/ml alginate in a 50 ml Falcon tube.
this mixture is allowed to cool on ice for at least 5 min

b) Encapsulation

The cells-alginate mixture described above is subsequently processed through the coaxial air flow device using the following settings:
flow rate pump: 0.5-1.5 ml/min
air flow meter: 2.5-3 L/min
pressure valve 1: 0.2 MPa
pressure valve 2: 0.1 MPa

These settings will vary (higher or lower) depending on the size of the particles one wants to produce.

Using a peristaltic pump the cell-alginate mixture is aspirated out of the 50 ml Falcon tube using a metal hub needle (gauge 16), and advanced through a tubing towards the 22 gauge air-jet needle. Upon extrusion through the 22 gauge air-jet needle droplets are produced by a combination of air shears and mechanical pressure by the peristaltic pump. Droplets containing islets in alginate are produced by extrusion (0.5-1.5 ml/min) through a 22 gauge air-jet needle (air flow 2.5-3 l/min).

Droplets fall 2 cm lower into a 20 ml beaker containing a solution of 50 mM CaCl₂ and 1 mM BaCl₂ (in 10 mM MOPS, 0.14 M mannitol and 0.05% Tween20, pH 7.2-7.4) as gelling solution. Upon contact with this buffer the microdroplets jellify (Qi et al.; 2008). Droplet size will vary between 200-800 μm, depending on pump flow rate and on air flow used.

The droplets are left for 7 minutes in the BaCl₂-gelling solution. Afterwards the capsules are removed from the gelling solution by pouring this capsules containing gelling solution over a cylinder shaped sieve with a 22 mesh grid at the bottom.

Afterwards capsules are gently washed by dipping the cylinder shaped sieve containing the particles repeatedly in a glass recipient filled with Ringer's or Hanks Balanced Salt Solution. This step is repeated three times with each time a complete renewal of the washing solution.

After taking samples for QC, capsules are cultured in albumin free or albumin containing or Ham F-10 medium at 37° C. and 5% CO₂ until transplantation

Alternatively an electrostatic bead generator can be used to produce the droplets.

c) Transplantation and results: normalization after transplantation

Diabetes was induced in immune-deficient Nod/Scid mice by treatment with 50 mg/kg Alloxan monohydrate (2,4,5,6-tetraoxypyrimidine; 2,4,5,6-pyrimidinetetrone, a glucose analog). Animals were monitored for a stable diabetic state prior to entry into the study. As a control, a healthy mouse was used. Transplantations were performed 2 days after alloxan treatment. Five animals were implanted with 2.9 million alginate encapsulated human beta cells/animal in the peritoneal cavity (19M beta cells/ml of alginate). A small incision was made in the abdominal wall and peritoneum of the animal along the linea alba. Encapsulated cells were subsequently transferred into the peritoneal cavity using a 5 ml pipette filled with 4 ml buffer solution. Two diabetic animals received no implantation. The animals were then monitored for up to 258 days. Blood glucose measurements were taken under non-fasting conditions. The experiment was split into three experimental groups:
Group 1 (depicted with triangles in the figures): diabetic mice implanted with encapsulated human beta cells in the peritoneal cavity (n=5)
Group 2 (depicted with squares in the figures): diabetic mice, which were not transplanted with human beta cells (n=2)
Group 3 (depicted with diamonds in the figures): non-diabetic mouse, negative control (n=1). Only one animal was included in this group as there is sufficient historical data for this group.

Blood was drawn from the animals to measure blood glucose, C-peptide and pro-insulin levels. The body weight of the animals was also measured. Following the sacrifice of the animals (at week five and 37) free floating capsules were retrieved and both the cells and the capsules were analyzed using light microscopy (H&E), semi-thin section, ultra-thin section and electron microscopy to determine cell viability, insulin production and glucagon production.

Electron microscopy was used to estimate cell viability (by counting 1000 cells) and showed that post-encapsulation the viability was 81%, compared to 88% for the non-encapsulated cells. Viability was also measured just prior to implantation and was found to be 62% compared with non-encapsulated cells treated in a similar fashion that showed 94% viability. The average diameter of the capsules was 620 μm, prior to implantation. Following sacrifice of animals, at both day 35 and 258, the majority of the capsules were found to be free floating in the peritoneal cavity and were collected by flushing the cavity. There was a slight reduction in the size of the capsules following implantation with a 7 and 8% reduction in the capsules diameter at days 35 and 258, respectively. The percentage of viable cells appeared to vary significantly between animals, but was always greater than 57% even after 258 days. Even though the percentage of viable cells varied, the percentage of insulin and glucagon positive cells remained more constant at 55 and 15.5%, respectively. It was not possible to quantify the total number of encapsulated cells.

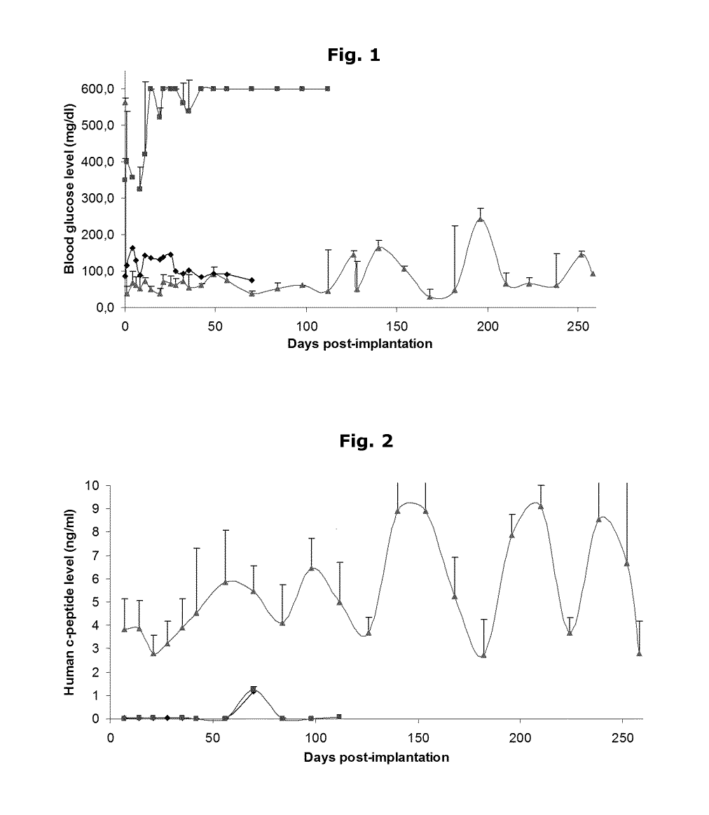

Prior to implantation both the diabetic groups (group 1 & 2) showed high levels of blood glucose compared to the non-diabetic control (group 3). This is characteristic of the loss of glucose control observed in diabetic patients. The first post-implantation blood glucose measurement was performed at 24 hours and showed that in all five animals of group 2 (treated with encapsulated human beta cells) showed a highly significant decrease in blood glucose to a level comparable to that seen for the normal non-diabetic control (FIG. 1). The normalization of blood glucose was maintained during a period of at least 110 days. After this initial period a variation in blood glucose levels was observed between animals and between the time points, suggesting that therapeutic advantage of the human beta cells was gradually being lost. Blood glucose levels, however, remained significantly lower than that of the diabetic controls (group 2). For the diabetic animals that were not implanted with human beta cells the non-fasting blood glucose levels remained high.

To further characterise the normalisation of blood glucose levels, the level of circulating human C-peptide and human pro-insulin were monitored. The assays used are able to differentiate human from rodent oligopeptides and therefore provides a direct measure of the functionality of the human beta cells. Circulating human C-peptide is detected at the initial time point tested (one week) in all five animals implanted with encapsulated human beta cells. There appears to be a gradual increase in C-peptide over the first eight weeks post-implantation. The level of circulating human C-peptide shows significant fluctuation over the remainder of the study remains above 3 ng/ml. This data is consistent with the blood glucose data in 2. No C-peptide was detected in the mice that were not implanted with human beta cells. This confirms the specificity of the test for human C-peptide. The level of human C-peptide observed in this experiment is considered to be physiologically relevant as they are above the level of circulating human C-peptide in normal healthy humans (0.9-1.8 ng/ml).

Similar data is seen when characterising the circulating level of human pro-insulin. All five diabetic animals treated with human beta cells show quantifiable levels of pro-insulin at the first one week time point. Only group 1 animals, containing implanted encapsulated human cells, show consistent pro-insulin expression above the detection limit of the test (greater than 14 pmol/l throughout the duration of the study) (FIG. 2).

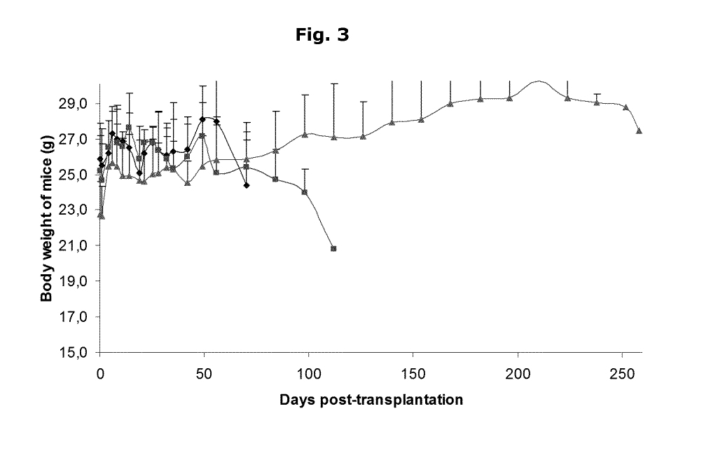

The body weight of the animals was also monitored throughout the study in order to measure any toxicity associated with the diabetic state and/or the treatment (FIG. 3). All animals treated with encapsulated human beta cells (group 1) maintained or even slightly increased their body weight suggesting that there were no toxic effects associated with the implantation. The non-treated diabetic group (group 2) maintained body weight for the majority of the study but showed a decrease in body weight later in the study, which was associated with the diabetic pathology. Surprisingly the normal control animal (group 3) showed a decrease in weight early in the study and was excluded. This has not been previously observed in historical data and is considered to be unrelated to this experiment. No other signs of adverse events were observed within this study.

### Example2

### Encapsulation of Cells in Alginate Filaments

Human or porcine beta cells are mixed with alginate 1.8% using a pipet until homogeneous suspension is obtained. Human islets are mixed with a 1.8% sterile ultrapure alginate solution to obtain a final cell density between 5-50×10⁶ cells/ml alginate in a 50 mL Falcon tube. This mixture is allowed to cool on ice for at least 5 min Using a peristaltic pump the cell-alginate mixture is subsequently aspirated out of the 50 ml Falcon tube using a metal hub needle (gauge 16), and advanced through a tubing towards the 22 gauge needle. The tip of the needle is placed in the gelling solution.

Upon extrusion through the 22 gauge needle the alginate immediately makes contact with the gelling solution (50 mM CaCl₂ and 1 mM BaCl₂ in 10 mM MOPS, 0.14 M mannitol and 0.05% Tween20, pH 7.2-7.4) immediately forming a cylindrical filament containing cells. Uninterrupted filaments of several meters long can thus be generated.

In order to obtain a smooth surface of the filaments preferably a tall beaker (preferably more than 20 cm high) is used as recipient for the gelling solution.

The diameter of the filaments can vary between 50-1200 μm, depending on pump flow rate and on the gauge or inner diameter of the needle used. Preferably the diameter of the filament is kept below 800 μm in order not to negatively influence the exchange of nutrients and gasses with the environment.

The filaments are left for 7 minutes in the BaCl₂-gelling solution. Afterwards the filaments are removed from the gelling solution by pouring this filaments containing gelling solution over a cylinder shaped sieve with a 22 mesh grid at the bottom

Afterwards filaments are gently washed by dipping the cylinder shaped sieve containing the filaments repeatedly in a glass recipient filled with Ringer's or Hanks Balanced Salt Solution. This step is repeated three times with each time a complete renewal of the washing solution.

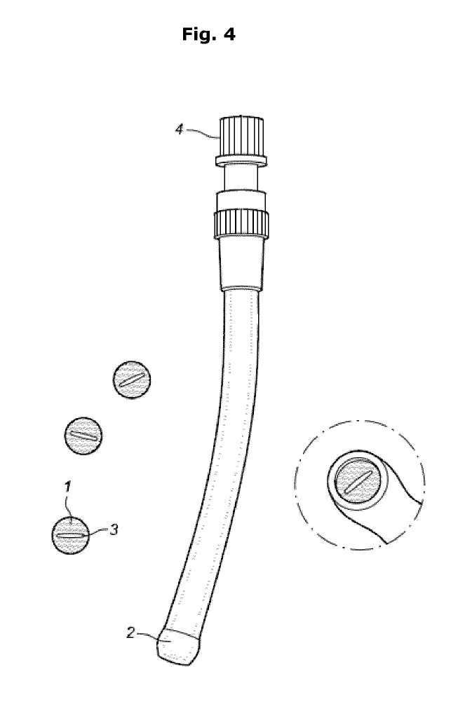

After taking samples for QC, particles are cultured in albumin free or albumin containing or Ham F-10 medium at 37° C. and 5% CO₂ until transplantation. Instead of a needle an “in house” developed nozzle can be used (FIG. 4). This nozzle consists out of a cylindrical plastic or plexi-glass piece (1), which can be inserted in the tail-end of tubing (2). With a laser a rectangular or egg shape hole (3) has been burned through this plastic or plexi-glass piece. When the tip of the tubing (containing the plexi or plastic nozzle) is placed below the surface of the barium/calcium gelling buffer and when the alginate or a cell-alginate mixture is pushed through this nozzle piece (4) (using a peristaltic pump) also filaments can be produced. The shape of the filaments will vary from cylindrical to sheet (beam) like, depending on the width of the laser made perforation in the piece.

There are advantages inherent to the filamentous shape itself: they can be more easily handled and surgically or laparoscopically transplanted in sites other than the peritoneum such as, but not limited to fat, omentum, subcutane. In case of clinical complications they might also be easier removed than the common alginate capsules.

### Example 3

### Generation of Double Walled Capsules by Consecutive Rounds of Encapsulation

Cells can be encapsulated in double walled alginate capsules. Doing so, cells or cell clusters trapped near or in the wall of the capsule after the first round of encapsulation will be covered by a second layer of alginate during the second round of encapsulation. By doing so, the exposure of encapsulated cells directly to the body will be even more limited. A direct immune response towards cells extruding from the capsule after a single round of encapsulation can thus be excluded.

In a first round of encapsulation cells will be encapsulated as follows: using a peristaltic pump the cell-alginate mixture is aspirated out of the 50 ml Falcon tube using a metal hub needle (gauge 16), and advanced through a tubing towards the 25 gauge air-jet needle. Upon extrusion through the 25 gauge air-jet needle droplets are produced by a combination of air shears and mechanical pressure by the peristaltic pump. Droplets containing islets in alginate are produced by extrusion (1.2-1.5 ml/min) through a 22 gauge air-jet needle (air flow 2,5-3 l/min).

Droplets fall 2 cm lower into a 20 ml beaker containing 50 mM CaCl₂ and 1 mM BaCl₂ (in 10 mM MOPS, 0.14 M mannitol and 0.05% Tween20, pH 7.2-7.4) as gelling solution. Upon contact with this buffer the microdroplets jellify (Qi et al.; 2008). Particle size will vary between 200-800 μm, depending on pump flow rate and on air flow used.

The particles are left for 7 minutes in the BaCl₂-solution. After the particles are removed from the gelling solution by pouring this particles containing gelling solution over a cylinder shaped sieve with a 22 mesh grid at the bottom

Afterwards particles are gently washed by dipping the cylinder shaped sieve containing the particles repeatedly in a glass recipient filled with Ringer's or Hanks Balanced Salt Solution. This step is repeated three times with each time a complete renewal of the washing solution.

The capsules obtained this way will subsequently undergo a second round of encapsulation. Capsules generated during the first round of encapsulation will therefore be mixed again with alginate 1.8% using a pipet until homogeneous suspension is obtained.

The second round of encapsulation is done in a similar way as the first with the exception that for the second round of encapsulation the alginate plus particles mixture is extruded through a 22 gauge needle.

The gauge size of the needles is not restricted to the combination (25 gauge and 22 gauge) utilized above. The diameter of the particles produced after the first encapsulation round and the thickness of the second alginate layer (generated during the second encapsulation round) are largely determined by the inner diameter of both needles.

The alginate used during the first encapsulation round can be high G-alginate or high M-alginate. The alginate used during the second encapsulation round can be high G-alginate or high M-alginate.

The alginate concentration during the first and second encapsulation round can vary between 1.4 and 2 percent.

### Example 4

### Maturation of Cells in Alginate Matrices

Perinatal porcine islets could be encapsulated in alginate matrices containing the basement membrane proteins collagen type IV and laminin, individually and in combination, at a total protein concentration of 10-200 μg/ml. It can be expected that islet insulin secretion will be increased compared to islets encapsulated in alginate particles without these basement membrane proteins

Alginate conjugates can include, but are not limited to, alginate-collagen, alginate-laminin, alginate-elastin, alginate-fibronectin, alginate-collagen-laminin and alginate-hyaluronic acid in which the collagen, laminin, elastin, collagen-laminin or hyaluronic acid is covalently bonded (or not bonded) to alginate. Examples of salts which can be used to gel the alginate constructs include, but are not limited to, calcium chloride (CaCl₂), barium chloride (BaCl₂) or strontium chloride (SrCl₂). Laminin and collagen type I could increase accumulated insulin release, while fibronectin could result in increased cell proliferation.

### Example 5

### Encapsulation of Beta Cells and Adipocytes to Improve Functionality

Results have shown that transplantation in fat tissue might be beneficial for the functionality of beta cells. Chen et al. (2009) showed that streptozotocin-induced diabetic FVB/NJ mice could be rendered normoglycemic with a therapeutic mass of syngeneic islets implanted in the epididymal fat pad, followed by a subrenal capsular implantation of a subtherapeutic mass of 25 islets from young (3 months) or old (24 months) mice. Three weeks after the second transplant, the islet containing fat pad was removed to reintroduce hyperglycemia.

Adipocytes can be prepared from white epididymal fat pads after tissue dissociation with collagenase digestion, filtration through 150-μm nylon membrane, and centrifugation (5 min, 300 rpm). Isolated adipocytes can be cultured in minimum DMEM medium (Life Technologies) supplemented with streptomycin/penicillin (100 μg/ml each) at 37° C.

Mixtures of different percentages of beta cells and freshly isolated or cultured adipocytes can subsequently be encapsulated in 1.8% sterile ultrapure alginate solution to obtain a final cell density between 5-50×10⁶ cells/ml alginate. Doing so, the adipocytes which were co-encapsulated with the beta cells can provide the proper matrix for the beta cells and initiate or stimulate the functionality of these encapsulated beta cells in vivo.

1. A method for encapsulating biological material, the method comprising:
forming a mixture of the biological material with a biocompatible matrix composition,
providing the mixture to a solution comprising calcium and barium cationic cross-linking agents,
forming micro-droplets in the mixture by the jellification of the biocompatible matrix composition, and
rinsing the micro-droplets in an aqueous buffer and maintaining the micro-droplets in a serum-free nutrient buffer.

2. The method according to claim 1, whereby the biocompatible matrix composition is selected from the group consisting of agar, alginate, carrageenan, cellulose and its derivatives, chitosan, collagen, gelatin, epoxy resin, photo cross-linkable resins, polyacrylamide, polyester, polystyrene or polyurethane, and polyethylene glycol.

3. The method according to claim 1, wherein the micro-droplets are shaped as granules, spheres or filaments.

4. The method according to claim 1, wherein the micro-droplets are sized between 200 and 800 μm.

5. The method according to claim 1, wherein the biological material comprises DNA, RNA, organelles, hoiniones, viable tissue, viable cells, proteins, antibodies, immuno-proteins, and/or peptides.

6. The method according to claim 5, wherein the biological material comprises viable cells.

7. The method according to claim 6, wherein the cells are selected from the group consisting of islet cells, hepatocytes, neuronal cells, pituitary cells, chromaffin cells, chondrocytes and any other cell type that is able to secrete factors, or insulin.

8. The method according to claim 6, whereby the micro-droplets comprise a cell density between 10×10⁶ and 30×10⁶ cells per ml alginate.

9. An encapsulated biological material produced by the method of claim 1.

10. The encapsulated biological material of claim 9, in a filamentous form.

11. A therapeutic agent comprising the encapsulated biological material of claim 9 suitable for ameliorating or treating a condition in an animal, including a human.

12. The encapsulated biological material of claim 9 in implantable, transplantable, or injectable form.

13. The encapsulated biological material of claim 9, wherein the biological material comprises pancreatic endocrine cells of mammalian origin.

14. The encapsulated biological material of claim 13, wherein the pancreatic endocrine cells originate from immature porcine pancreas.

15. An implantation or transplantation material comprising the encapsulated biological material of claim 9, wherein the implantation or transplantation site is selected from the group consisting of subcutaneous, intramuscular, intra-organ, intravenous, arterial/venous vascularity of an organ, cerebrospinal fluid, and lymphatic fluid.

16. The method according to claim 1, wherein the biocompatible matrix composition comprises at alginate.

17. The method according to claim 6, wherein the viable cells a selected from the group consisting of mammalian cells, progenitor and progenitor-derived cells, stem cells or stem cell-derived cells, and genetically engineered cells.

18. The therapeutic agent of claim 11, wherein the condition to be treated or ameliorated is diabetes.

19. The encapsulated biological material of claim 14, wherein pancreatic endocrine cells secrete insulin.

20. A method for ameliorating or treating a disease or condition in an animal, including a human, the method comprising:
transplanting an effective amount of a cell-containing alginate into said animal,
wherein the cells in the cell-containing alginate secrete a therapeutic substance that is effective in ameliorating or treating said disease or condition in said animal, and
wherein the cells are perinatal porcine islet cells and wherein the alginate is a high G-alginate or a high-M alginate.

21. The method according to claim 20, wherein the perinatal porcine islet cells are beta cells.

22. The method according to claim 20, wherein the cell-containing alginate is transplanted into the peritoneal cavity.

23. The method according to claim 22 wherein the cell-containing alginate is transplanted into the omentum.

24. The method according to claim 20, wherein the cell-containing alginate comprises between 1.4 and 2 percent alginate.

25. The method according to claim 20 wherein the therapeutic substance is insulin.
